(19) United States
(12) Patent Application Publication
(10) Pub. No.: US 2021/0139910 A1
(43) Pub. Date: May 13, 2021

(54) METHOD OF TREATING A MAMMAL WITH DIABETES ASSOCIATED KIDNEY DISEASE USING LOCAL ADMINISTRATION OF STEM CELLS WITH TRANSIENTLY REDUCED p53

(71) Applicant: The George Washington University, Washington, DC (US)

(72) Inventor: Sabyasachi SEN, Bethesda, MD (US)

(21) Appl. No.: 17/062,362

(22) Filed: Oct. 2, 2020

Related U.S. Application Data
(63) Continuation of application No. PCT/US19/26132, filed on Apr. 5, 2019.
(60) Provisional application No. 62/653,401, filed on Apr. 5, 2018.

(51) Int. Cl. C12N 15/113; A61K 35/28; A61K 35/51; A61K 35/14; A61K 38/44; A61K 9/00; A61P 13/12; A61P 3/10; A61K 35/44

(52) U.S. Cl. CPC C12N 15/1135; A61K 48/00; A61K 35/51; A61K 35/14; A61K 38/446; C12Y 115/01001; A61K 9/0024; A61P 13/12; A61P 3/10; A61K 9/0019; A61K 35/44; C12N 2310/531; C12N 2310/14; C12N 2320/32; A61K 35/28

(57) ABSTRACT

A composition comprising an endothelial progenitor cell genetically modified to have transiently reduced p53 expression for treating diabetic kidney disease and uses thereof

### CROSS REFERENCE TO RELATED APPLICATION

This application is a continuation of PCT Application No. PCT/US19/26132, filed Apr. 5, 2019, for “METHOD OF TREATING A MAMMAL WITH DIABETES ASSOCIATED KIDNEY DISEASE USING LOCAL ADMINISTRATION OF STEM CELLS WITH TRANSIENTLY REDUCED p53,” which claims the benefit of U.S. Provisional Application No. 62/653,401, filed on Apr. 5, 2018, the disclosures of which are hereby incorporated by reference.

### STATEMENT REGARDING SEQUENCE LISTING

The instant application contains a Sequence Listing which has been submitted via ASCII copy created on Oct. 1, 2020 referred to as ‘GW068_Sequence Listing_ST25.txt” and is 818 bytes having 2 sequences.

### FIELD

The present disclosure generally relates to compositions and methods for the treatment of diabetes associated kidney disease using endothelial lineage stem cell therapy, specifically by local administration of endothelial progenitor cells with transiently reduced p53.

### BACKGROUND

Therapeutic applications of induced pluripotent stem cells are of great interest to many fields of medicine; however, current therapies are limited to readily available endogenous progenitor cell populations that can be isolated or mobilized from peripheral blood and/or bone marrow. These include hematopoietic stem cells (HSCs), mesenchymal stem cells (MSCs), and endothelial progenitor cells (EPCs). Currently, clinical application of endothelial lineage stem cell therapies remains difficult due to the low quantity and quality of endogenous progenitor cells isolated from bone marrow and peripheral blood of certain patient populations.

A patient population that could greatly benefit from endothelial lineage stem cell therapies are those with diabetic kidney disease, or diabetic nephropathy. Diabetic nephropathy is an early vascular complication with poor renal outcomes in patients with type 1 diabetes mellitus (DM1) or type 2 diabetes mellitus (DM2). Other than kidney transplantation, no definitive therapy has been successful at treating diabetic nephropathy or improving the renal symptoms associated with diabetic nephropathy.

### SUMMARY

In an aspect, the disclosure provides a composition comprising an endothelial progenitor cell genetically modified to have transiently reduced p53 expression for treating diabetic kidney disease. The genetically modified endothelial progenitor cell comprises transiently reduced p53 expression for at least 4 weeks. The composition further comprises at least one pharmaceutically acceptable excipient.

The endothelial progenitor cells can be obtained from peripheral blood, umbilical cord blood, or bone marrow of a subject. In some instances, the endothelial progenitor cell obtained is autologous to the subject. In other instances, the endothelial progenitor cell obtained is allogeneic to the subject. Specifically, endothelial progenitor cells are derived from human CD34⁺ mononuclear cells.

The endothelial progenitor cell genetically modified to have transiently reduced p53 expression is also genetically modified to have transiently increased expression of at least one mitochondrial antioxidant. Specifically, the endothelial progenitor cell is genetically modified to have transiently reduced p53 expression is also genetically modified to have transiently increased manganese superoxide dismutase (MnSOD) expression.

In another aspect, the disclosure provides a composition for transplant under a kidney capsule comprising an endothelial progenitor cell genetically modified to have transiently reduced p53 expression. The genetically modified endothelial progenitor cell comprises transiently reduced p53 expression for at least 4 weeks. The composition further comprises at least one pharmaceutically acceptable excipient.

In yet another aspect, the disclosure provides a method of treating diabetic kidney disease in a subject in need thereof, the method comprising transplanting an endothelial progenitor cell genetically modified to have transiently reduced p53 expression under at least one of the subject's kidney capsule. The method can be performed at least once a month.

The subject in need thereof has type 1 diabetes mellitus, type 2 diabetes mellitus, or is pre-diabetic. The subject in need thereof has at least one symptom of diabetic kidney disease. The symptoms of diabetic kidney disease can be is proteinuria, renal fibrosis, at least a 25% decrease in estimated glomerular filtration rate (eGFR) compared to eGFR of a non-diabetic subject, at least a 25% decrease in urinary creatinine clearance compared to urinary creatinine clearance of a non-diabetic subject, at least a 25% decrease in renal blood flow compared to renal blood flow of a non-diabetic subject, at least a 25% loss of podocytes compared to the amount of podocytes of a non-diabetic subject, or a combination thereof. The method disclosed in this aspect increases diabetic life expectancy of the treated subject by at least 5% compared to an untreated subject with identical disease condition and predicted outcome.

In yet another aspect, the disclosure provides a method of treating at least one symptom of diabetic kidney disease, the method comprising transplanting an endothelial progenitor cell genetically modified to have transiently reduced p53 expression under at least one kidney capsule of a subject in need thereof. Where the symptom of diabetic kidney disease is proteinuria, the method disclosed in this aspect improves proteinuria by at least 25% within one month after transplanting an endothelial progenitor cell genetically modified to have transiently reduced p53 expression under at least one kidney capsule. Where the symptom of diabetic kidney disease is renal fibrosis, the method disclosed in this aspect decreases renal fibrosis by at least 1% within one month after transplanting an endothelial progenitor cell genetically modified to have transiently reduced p53 expression under at least one kidney capsule. Where the symptom of diabetic kidney disease is at least 25% decrease in estimated glomerular filtration rate (eGFR) compared to eGFR of a non-diabetic subject, the method disclosed in this aspect increases eGFR by at least 10% within one month after transplanting an endothelial progenitor cell genetically modified to have transiently reduced p53 expression under at least one kidney capsule. Where the symptom of diabetic kidney disease is at least 25% decrease in urinary creatinine clearance compared to urinary creatinine clearance of a non-diabetic subject, the method disclosed in this aspect increases urinary creatinine clearance by at least 25% within one month after transplanting an endothelial progenitor cell genetically modified to have transiently reduced p53 expression under at least one kidney capsule. Where the symptom of diabetic kidney disease is at least 25% decrease in renal blood flow compared to renal blood flow of a non-diabetic subject, the method disclosed in this aspect increases renal blood flow by at least 5% within one month after transplanting an endothelial progenitor cell genetically modified to have transiently reduced p53 expression under at least one kidney capsule. Where the symptom of diabetic kidney disease is at least 25% loss of podocytes compared to podocyte amount of a non-diabetic subject, the method disclosed in this aspect decreases the rate of podocyte loss within one month after transplanting an endothelial progenitor cell genetically modified to have transiently reduced p53 expression under at least one kidney capsule.

The method disclosed in this aspect increases the expression of at least one anti-oxidant marker wherein the anti-oxidant marker comprises at least one selected from the group of SOD1 (superoxide dismutase 1), SOD2 (superoxide dismutase 2), CAT (catalase), GPX1 (glutathione peroxidase 1), and GPX3 (Glutathione peroxidase 3). The method disclosed in this aspect also increases the expression of at least one angiogenesis marker wherein the angiogenesis marker comprises at least one selected from the group of VEGF-A (vascular endothelial growth factor-A), PECAM1 (Platelet And Endothelial Cell Adhesion Molecule 1), eNOS (endothelial NOS), and KDR (vascular endothelial growth factor receptor 2).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts a graph showing urinary protein excretion from (1) healthy mice (no STZ), (2) mice with diabetic kidney disease transplanted with endothelial progenitor cells (EPCs) bilaterally under the kidney capsule (null), and (3) mice with diabetic kidney disease transplanted with p53-silenced EPCs bilaterally under the kidney capsule (p53sh). Protein was measured from 24-hour urine collections taken before EPC transplantation (week 0) and weeks 1, 2, 3, and 4 after EPC transplantation with a Randox RX Monza chemical analyzer.

FIG. 2A depicts a graph showing urinary creatinine from (1) healthy mice (no STZ), (2) mice with diabetic kidney disease transplanted with endothelial progenitor cells (EPCs) bilaterally under the kidney capsule (null), and (3) mice with diabetic kidney disease transplanted with p53-silenced EPCs bilaterally under the kidney capsule (p53sh). Creatinine was measured from 24-hour urine collections taken before EPC transplantation (week 0) and weeks 1, 2, 3, and 4 after EPC transplantation with a Randox RX Monza chemical analyzer.

FIG. 2B depicts a bar graph showing plasma creatinine levels of (1) healthy mice (no STZ), (2) mice with diabetic kidney disease transplanted with endothelial progenitor cells (EPCs) bilaterally under the kidney capsule (null), and (3) mice with diabetic kidney disease transplanted with p53-silenced EPCs bilaterally under the kidney capsule (p53sh). Creatinine was measured 4 weeks after EPC transplantation with a Randox RX Monza chemical analyzer.

FIG. 3 depicts a bar graph showing renal blood flow of (1) mice with diabetic kidney disease transplanted with endothelial progenitor cells (EPCs) bilaterally under the kidney capsule (null) and (2) mice with diabetic kidney disease transplanted with p53-silenced EPCs bilaterally under the kidney capsule (p53sh). Renal blood flow was measured 28 days after EPC transplantation with a Laser Doppler Perfusion Imager (LDPI) system.

FIG. 4A depicts a graph showing the area under the curve (AUC) for urinary protein excretion from mice with diabetic kidney disease transplanted with either (1) EPCs bilaterally under the kidney capsule (null), (2) p53-silenced EPCs bilaterally under the kidney capsule (p53 sh), or (3) mesenchymal stromal cells (MSCs) bilaterally under the kidney capsule. AUC was plotted from urinary protein measurements from 24-hour urine collections taken before transplantation (week 0) and weeks 1, 2, 3, and 4 after transplantation with a Randox RX Monza chemical analyzer.

FIG. 4B depicts a graph showing the area under the curve (AUC) for the ratio of urinary protein to urinary creatinine from mice with diabetic kidney disease transplanted with either (1) EPCs bilaterally under the kidney capsule (null), (2) p53-silenced EPCs bilaterally under the kidney capsule (p53 sh), or (3) mesenchymal stromal cells (MSCs) bilaterally under the kidney capsule. AUC was plotted from urinary protein and urinary creatinine measurements from 24-hour urine collections taken before transplantation (week 0) and weeks 1, 2, 3, and 4 after transplantation with a Randox RX Monza chemical analyzer.

FIG. 4C depicts a bar graph showing renal blood flow of healthy, sham-treated mice in ratio to mice with diabetic kidney disease transplanted with either (1) EPCs bilaterally under the kidney capsule (null), (2) p53-silenced EPCs bilaterally under the kidney capsule (p53sh), or (3) mesenchymal stromal cells (MSCs) bilaterally under the kidney capsule. Renal blood flow was measured 28 days after sham-treatment or transplantation with a Laser Doppler Perfusion Imager (LDPI) system.

FIG. 5A depicts an image of a kidney section stained with isolectin-β4 (brown) and hematoxylin (blue) from a diabetic, sham-treated mouse.

FIG. 5B depicts an image of a kidney section stained with isolectin-β4 (brown) and hematoxylin (blue) from a mouse with diabetic kidney disease 28 days after EPCs were transplanted bilaterally under the kidney capsule.

FIG. 5C depicts an image of a kidney section stained with isolectin-β4 (brown) and hematoxylin (blue) from a mouse with diabetic kidney disease 28 days after p53-silenced EPCs were transplanted bilaterally under the kidney capsule.

FIG. 5D depicts an image of a kidney section stained with isolectin-β4 (brown) and hematoxylin (blue) from a mouse with diabetic kidney disease 28 days after MSCs were transplanted bilaterally under the kidney capsule.

FIG. 6A depicts a bar graph showing mRNA gene expression levels of the vascular markers eNOS, VEGF-A and KDR as measured in the kidney tissue of (1) mice with diabetic kidney disease transplanted with endothelial progenitor cells (EPCs) bilaterally under the kidney capsule (null) and (2) mice with diabetic kidney disease transplanted with p53-silenced EPCs bilaterally under the kidney capsule (p53sh). Gene expression levels were measured 28 days after EPC transplantation by qRT-PCR.

FIG. 6B depicts a bar graph showing mRNA gene expression levels of the vascular markers eNOS, VEGF-A and KDR as measured in the kidney tissue of mice with diabetic kidney disease transplanted with either (1) EPCs bilaterally under the kidney capsule (null), (2) p53-silenced EPCs bilaterally under the kidney capsule (p53sh), or (3) mesenchymal stromal cells (MSCs) bilaterally under the kidney capsule. Gene expression levels were measured 28 days after transplantation by qRT-PCR.

FIG. 7 depicts a bar graph showing mRNA gene expression levels of anti-oxidant markers (SOD1, SOD2, CAT, GPX1, and GPX3) and angiogenesis markers (N053, KDR, VEGF-A, PECAM1) in mature human, endothelial cells (HUVECS) either (1) exposed to high glucose (20 mM) for 28 days (HG) or (2) not exposed to glucose (NG). Gene expression levels were measured by qRT-PCR.

### DETAILED DESCRIPTION

Compositions comprised of at least an endothelial progenitor cell directed toward treating diabetic kidney disease detailed below. As used herein, the term “endothelial progenitor cell” refers to a precursor cell that is present in blood such as mammalian peripheral blood, bone marrow, and cord blood. In general, the compositions disclosed herein comprise at least one endothelial progenitor cell genetically modified to have transiently reduced p53 expression. In various embodiments, compositions of the present disclosure further comprise at least one pharmaceutically acceptable excipient. In various embodiments, compositions of the present disclosure may be used to treat diabetic kidney disease and/or at least one symptom associated with diabetic kidney disease.

In treating with diabetic kidney disease, or diabetic nephropathy, improved vascularization and/or re-perfusion of a diabetic sclerotic kidney is a goal of a developing therapy. Accordingly, an endothelial lineage stem or progenitor cell may be a beneficial treatment for reversing the kidney damage attributed to diabetic nephropathy.

A complicating factor, however, is that endogenous progenitor cells are susceptible to apoptosis in a hyperglycemic environment. As such, isolation of functional endogenous progenitor cells from the bone marrow and/or peripheral blood of a diabetic patient is difficult as cells will have increased susceptibility to apoptosis in the presence of the high blood glucose levels that occur with diabetes. Accordingly, the disclosure herein provides for improved efficacy of endothelial lineage stem cell therapies, particularly for use in the treatment of diabetic nephropathy. As disclosed herein methodological approaches' herein enhance endogenous progenitor cell survival after isolation from a diabetic patient and increase endogenous progenitor cell longevity after administration to a diabetic patient.

### (I) Compositions

Aspects of the present disclosure encompass a composition comprising an endothelial progenitor cell genetically modified to have transiently reduced p53 expression. A composition disclosed herein may encompass an endothelial progenitor cell genetically modified to have transiently reduced p53 expression for at least four weeks. A composition disclosed herein may further comprise at least one pharmaceutically acceptable excipient. A composition disclosed herein may be a composition for treating diabetic kidney disease. As used herein, the term “treatment” refers to administration of a therapeutic substance effective to ameliorate symptoms associated with renal disease, to lessen the severity or cure the renal disease, or to prevent the disease from occurring or from spreading. A composition disclosed herein may be for transplant under a kidney capsule.

(a) Endothelial Progenitor Cells

In various embodiments, compositions disclosed herein comprise an endothelial progenitor cell genetically modified to have transiently reduced p53 expression. In various embodiments, an endothelial progenitor cell can be isolated from bone marrow. In other embodiments, an endothelial progenitor cell can be isolated from peripheral blood. In still other embodiments, an endothelial progenitor cell can be isolated from umbilical cord blood. In yet other embodiments, an endothelial progenitor cell can be isolated from autologous peripheral blood, umbilical cord blood, and/or bone marrow. As used herein, the term “autologous” refers to peripheral blood, umbilical cord blood, and/or bone marrow obtained from the same subject to be treated with the compositions disclosed herein. In other embodiments, an endothelial progenitor cell can be isolated from allogeneic peripheral blood, umbilical cord blood, and/or bone marrow. As used herein, the term “allogeneic” refers to peripheral blood, umbilical cord blood, and/or bone marrow obtained from a different subject of the same species as the subject to be treated with the compositions disclosed herein. In yet other embodiments, an endothelial progenitor cell can be derived from human CD34⁺ mononuclear cells. In other embodiments, endothelial progenitor cells can be obtained by ex vivo expansion following isolation from peripheral blood, umbilical cord blood, and/or bone marrow. In still other embodiments, endothelial progenitor cells can be expanded in vivo by administration of at least one recruitment growth factor to the subject prior to removing progenitor cells. Non-limiting examples of recruitment growth factors include interleukin 3 (IL-3) and granulocyte-macrophage colony stimulating factor (GM-CSF). In some aspects, administration of at least one recruitment growth factor to the subject can occur at least 1 hour to at least 2 hours prior to removing progenitor cells.

In various embodiments, an endothelial progenitor cell may be genetically modified to transiently alter expression of at least one protein. In some aspects, an endothelial progenitor cell can be genetically altered by any method resulting in the uptake and expression of a nucleic acid sequence by the cells. Non-limiting examples of suitable methods for genetically altering an endothelial progenitor cell include vectors, viral vectors, liposomes, naked DNA, adjuvant-assisted DNA, catheters, and gene guns. In some aspects, introduction of a nucleic acid sequence can be by standard techniques, including, but not limited to, infection, transfection, transduction, and transformation. In preferred aspects, an endothelial progenitor cell may be genetically modified by ex vivo transduction of a nucleic acid sequence into cells using an adenoviral vector.

In various embodiments, an endothelial progenitor cell may be genetically modified to transiently reduce p53 expression. In some aspects, a genetically modified endothelial progenitor cell may comprise p53 specific siRNA or p53-specific shRNA. In other aspects, the p53 specific siRNA or p53-specific shRNA may be provided by an adenoviral vector. In still other aspects, adenoviral vectors can express at least one transcripts of interest for a length of time that approximates the time it takes an endothelial progenitor cell to differentiate into its mature endothelial cell. In yet other aspects, adenoviral vectors can express at least one transcripts of interest for at least 2 weeks, at least 4 weeks, or at least 6 weeks. In some aspects, the p53 specific siRNA provided to a endothelial progenitor cell by an adenoviral vector may comprise an oligonucleotide with SEQ ID NO: 1 (5′ GATCCCCGACTCCAGTGGTAATCTACTTCAAGAGAGTAGATTACCACTGGAGTCTTT TTGGAAA 3′). In other aspects, the p53 specific siRNA provided to an endothelial progenitor cell by an adenoviral vector may comprise an oligonucleotide with SEQ ID NO: 2 (5′AGCTTTTCCAAAAAGACTCCAGTGGTAATCTACTCTCTTGAAGTAGATTACCACTG GAGTCGGG 3′). In still other aspects, vectors that do not integrate into the chromosomal DNA of an endothelial progenitor cell may be used to express p53-specific siRNA in an endothelial progenitor cell.

In various embodiments, an endothelial progenitor cell may be genetically modified to transiently reduce p53 expression completely. In some aspects, an endothelial progenitor cell may be genetically modified to transiently reduce p53 expression from about 5% to about 75% from the amount of p53 expressed in the cell before genetic modification. In other aspects, an endothelial progenitor cell may be genetically modified to transiently reduce p53 expression to about 5%, about 10%, about 25%, about 50%, or about 75% from the amount of p53 expressed in the cell before genetic modification. In other aspects, an endothelial progenitor cell is genetically modified to transiently reduce p53 expression to about 30% to about 50% from the amount of p53 expressed in the cell before genetic modification. In preferred aspects, an endothelial progenitor cell is genetically modified to transiently reduce p53 expression to about 30%, about 33%, about 36%, about 40%, about 45%, or about 50% from the amount of p53 expressed in the cell before genetic modification.

In various embodiments, an endothelial progenitor cell may be genetically modified to transiently reduce at least one anti-apoptosis factor completely. In some aspects, an endothelial progenitor cell may be genetically modified to transiently reduce at least one anti-apoptosis factor from about 5% to about 75% from the amount of anti-apoptosis factor expressed in the cell before genetic modification. In other aspects, an endothelial progenitor cell may be genetically modified to transiently reduce at least one anti-apoptosis factor to about 5%, about 10%, about 25%, about 50%, or about 75% from the amount of anti-apoptosis factor expressed in the cell before genetic modification. In other aspects, an endothelial progenitor cell is genetically modified to transiently reduce at least one anti-apoptosis factor to about 30% to about 50% from the amount of anti-apoptosis factor expressed in the cell before genetic modification. In preferred aspects, an endothelial progenitor cell is genetically modified to transiently reduce p53 expression to about 30%, about 33%, about 36%, about 40%, about 45%, or about 50% from the amount of p53 expressed in the cell before genetic modification.

In various embodiments, an endothelial progenitor cell may be genetically modified to transiently upregulate at least one mitochondrial antioxidant. In some aspects, an endothelial progenitor cell may be genetically modified to transiently upregulate at least one mitochondrial antioxidant from about 5% to about 75% more than the amount of mitochondrial antioxidant expressed in the cell before genetic modification. In other aspects, an endothelial progenitor cell may be genetically modified to transiently upregulate at least one mitochondrial antioxidant to about 5%, about 10%, about 25%, about 50%, or about 75% more than the amount of mitochondrial antioxidant expressed in the cell before genetic modification. In other aspects, an endothelial progenitor cell is genetically modified to transiently upregulate at least one mitochondrial antioxidant to about 30% to about 50% more than the amount of mitochondrial antioxidant expressed in the cell before genetic modification. In preferred aspects, an endothelial progenitor cell is genetically modified upregulate at least one mitochondrial antioxidant expressed the cell to about 30%, about 33%, about 36%, about 40%, about 45%, or about 50% more from the amount of mitochondrial antioxidant expressed in the cell before genetic modification.

In various embodiments, an endothelial progenitor cell may be genetically modified to transiently upregulate manganese superoxide dismutase (MnSOD). In some aspects, an endothelial progenitor cell may be genetically modified to transiently upregulate MnSOD expression from about 5% to about 75% more than the amount of MnSOD expressed in the cell before genetic modification. In other aspects, an endothelial progenitor cell may be genetically modified to transiently upregulate MnSOD expression to about 5%, about 10%, about 25%, about 50%, or about 75% more than the amount of MnSOD expressed in the cell before genetic modification. In other aspects, an endothelial progenitor cell is genetically modified to transiently upregulate MnSOD expression to about 30% to about 50% more than the amount of MnSOD expressed in the cell before genetic modification. . In preferred aspects, an endothelial progenitor cell is genetically modified upregulate MnSOD expression in the cell to about 30%, about 33%, about 36%, about 40%, about 45%, or about 50% more from the amount of MnSOD expressed in the cell before genetic modification.

In various embodiments, an endothelial progenitor cell may be genetically modified to transiently reduce p53 expression and transiently upregulate manganese superoxide dismutase (MnSOD). In some aspects, an endothelial progenitor cell may be genetically modified to transiently reduce p53 expression from about 5% to about 75% less than the amount of p53 expressed in the cell before genetic modification and transiently upregulate MnSOD from about 5% to about 75% more than the amount of MnSOD expressed in the cell before genetic modification. In other aspects, an endothelial progenitor cell may be genetically modified to transiently reduce p53 expression to about 5%, about 10%, about 25%, about 50%, or about 75% less than the amount of p53 expressed in the cell before genetic modification and transiently upregulate MnSOD to about 5%, about 10%, about 25%, about 50%, or about 75% more than the amount of MnSOD expressed in the cell before genetic modification. In preferred aspects, an endothelial progenitor cell is genetically modified to transiently reduce p53 expression to about 50% less than the amount of p53 expressed in the cell before genetic modification and transiently upregulate MnSOD expression to about 50% more than the amount of MnSOD expressed in the cell before genetic modification.

In various embodiments, an endothelial progenitor cell may be genetically modified to transiently reduce p53 expression for at least 2 weeks. In some aspects, an endothelial progenitor cell may be genetically modified to transiently reduce p53 expression for about 2 weeks to about 12 weeks. In other aspects, an endothelial progenitor cell may be genetically modified to transiently reduce p53 expression for about at least about 4 weeks.

In various embodiments, an endothelial progenitor cell may be genetically modified to transiently reduce at least one anti-apoptosis factor for at least 2 weeks. In some aspects, an endothelial progenitor cell may be genetically modified to transiently reduce at least one anti-apoptosis factor for about 2 weeks to about 12 weeks. In other aspects, an endothelial progenitor cell may be genetically modified to transiently reduce at least one anti-apoptosis factor for at least about4 weeks.

In various embodiments, an endothelial progenitor cell may be genetically modified to transiently up regulate at least one mitochondrial antioxidant for at least 2 weeks. In some aspects, an endothelial progenitor cell may be genetically modified to transiently upregulate at least one mitochondrial antioxidant for about 2 weeks to about 12 weeks. In other aspects, an endothelial progenitor cell may be genetically modified to transiently upregulate at least one mitochondrial antioxidant for at least about 4 weeks.

In various embodiments, an endothelial progenitor cell may be genetically modified to transiently up regulate MnSOD expression for at least 2 weeks. In some aspects, an endothelial progenitor cell may be genetically modified to transiently upregulate MnSOD expression for about 2 weeks to about 12 weeks. In other aspects, an endothelial progenitor cell may be genetically modified to transiently upregulate MnSOD expression for at least about 4 weeks. (b) Pharmaceutically acceptable carriers and excipients.

In various embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof

In various embodiments, compositions disclosed herein comprise a pharmaceutical composition comprised of at least one an endothelial progenitor cell genetically modified to have transiently reduced p53 expression. In other embodiments, compositions disclosed herein comprise a pharmaceutical composition comprised of at least one an endothelial progenitor cell genetically modified to have expression of at least one anti-apoptosis factor transiently reduced. In still other embodiments, compositions disclosed herein comprise a pharmaceutical composition comprised of at least one an endothelial progenitor cell genetically modified to have expression of at least one mitochondrial antioxidant transiently increased. In other embodiments, compositions disclosed herein comprise a pharmaceutical composition comprised of at least one an endothelial progenitor cell genetically modified to have transiently increased MnSOD expression.

In some embodiments, pharmaceutical compositions disclosed herein may be formulated in a conventional manner using one or more physiologically acceptable carriers including excipients and auxiliaries which can facilitate processing of active components into preparations which can be used pharmaceutically. In other embodiments, proper formulation of pharmaceutical compositions disclosed herein may be dependent upon the route of administration chosen. In an aspect, any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety for such disclosure.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition

In various embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In specific embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the invention. In preferred aspects, additives will be biocompatible, and will not be harsh, abrasive, or allergenic.

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

(c) Dosage Formulations

In some embodiments, pharmaceutical compositions disclosed herein may be formulated for parenteral administration by injection. In some aspects, parenteral administration by injection can be by bolus injection and/or continuous infusion. In various embodiments, pharmaceutical compositions disclosed herein that are formulations for injection may be presented in unit dosage form. In some aspects, a unit dosage form may be in ampoules and or in multi-dose containers. In other aspects, pharmaceutical compositions disclosed herein may be suspensions, solutions or emulsions in oily or aqueous vehicles. In still other aspects, pharmaceutical compositions disclosed herein may contain formulatory agents such as suspending, stabilizing and/or dispersing agents. In yet other aspects, pharmaceutical compositions disclosed herein may be presented in unit-dose or multi-dose containers. Non-limiting examples of unit-dose or multi-dose containers include sealed ampoules and vials. In an aspect, pharmaceutical compositions disclosed herein may be stored in powder form or in a freeze-dried (lyophilized) condition requiring only the addition of the sterile liquid carrier immediately prior to use. In other aspects, pharmaceutical compositions disclosed herein may be extemporaneous injection solutions and suspensions may be prepared from sterile powders, granules, tablets or a combination thereof In still other aspects, pharmaceutical compositions disclosed herein may be cryofrozen prior to storage. As used herein, “cryofrozen” refers to and/or describes cryopreservation biological samples frozen in a manner that maintains vitality and subsequently thawed out again as needed while maintaining vitality. In some aspects, pharmaceutical compositions disclosed herein may be cryofrozen and stored for up to 1 week, up to 4 weeks, up to 8 weeks, up to 16 weeks, up to 25 weeks, up to 50 weeks, up to 100 weeks, or up to 200 weeks while maintaining vitality.

In various embodiments, pharmaceutical compositions described herein for parenteral administration can include aqueous and non-aqueous (oily) sterile injection solutions of the compositions which may contain antioxidants, buffers, bacteriostats and solutes which render the formulation isotonic with the blood of the intended recipient; and aqueous and non-aqueous sterile suspensions which may include suspending agents and thickening agents. In some aspects, pharmaceutical compositions described herein may include lipophilic solvents or vehicles. Non-limiting examples of vehicles include fatty oils such as sesame oil, or synthetic fatty acid esters, such as ethyl oleate or triglycerides, or liposomes. In various embodiments, pharmaceutical compositions described herein may be aqueous injection suspensions. In some aspects, pharmaceutical compositions described herein may contain substances which increase the viscosity of the suspension, such as sodium carboxymethyl cellulose, sorbitol, or dextran. In other aspects, pharmaceutical compositions described herein may comprise suitable stabilizers or agents which increase the solubility of the enzymes and fining agents to allow for the preparation of highly concentrated solutions.

In various embodiments, pharmaceutical compositions described herein may be formulated as a depot preparation. In some aspects, a depot preparation may be administered by implantation (for example subcutaneously or intramuscularly) or by intramuscular injection. In other embodiments, pharmaceutical compositions described herein may be formulated as a depot preparation locally administered under the kidney capsule. In still other embodiments, pharmaceutical compositions described herein may be formulated for retrograde transurethral delivery. In an aspect, formulations for retrograde transurethral delivery can allow compositions disclosed herein move upwards towards renal calyxes after giving at least one saline flush.

### (II) Uses of Compositions

In various embodiments, compositions disclosed herein may be effective for treating diabetic kidney disease following administration to a subject in need. In other embodiments, compositions disclosed herein may be effective for improving at least one symptom of diabetic kidney disease following administration to a subject in need.

A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In various embodiments, a subject in need may have been diagnosed with type 1 diabetes mellitus, type 2 diabetes mellitus, or is pre-diabetic. In some aspects, the subject may have diabetic kidney disease. In one embodiment, a subject may have diabetes-induced end stage renal failure. In another embodiment, a subject may at least one symptom of diabetic kidney disease. In some aspects, a symptom of diabetic kidney disease can be proteinuria. In yet other aspects, a symptom of diabetic kidney disease can be renal fibrosis. In other aspects, a symptom of diabetic kidney disease can be at least about a 25% decrease in estimated glomerular filtration rate (eGFR) compared to eGFR of a non-diabetic subject. In still other aspects, a symptom of diabetic kidney disease can be at least about a 25% decrease in urinary creatinine clearance compared to urinary creatinine clearance of a non-diabetic subject. In yet other aspects, a symptom of diabetic kidney disease can be at least about a 25% decrease in renal blood flow compared to renal blood flow of a non-diabetic subject. In other aspects, a symptom of diabetic kidney disease can be at least about a 25% to about a 50% loss of podocytes compared to the amount of podocytes of a non-diabetic subject.

Compositions disclosed herein may decrease diabetes-induced proteinuria compared to diabetes-induced proteinuria in an untreated subject with identical disease condition and predicted outcome. In some aspects, diabetes-induced proteinuria can be deceased by about by about 25% to about 50%, following administration of compositions disclosed herein.

Compositions disclosed herein may decrease and/or reverse diabetes-induced renal fibrosis compared to diabetes-induced renal fibrosis in an untreated diabetic subject with identical disease condition and predicted outcome. In some aspects, diabetes-induced renal fibrosis can be deceased by about 20% to about 40% following administration of compositions disclosed herein.

Compositions disclosed herein may reverse the diabetes-induced decline in eGFR compared to declining eGFR in an untreated diabetic subject with identical disease condition and predicted outcome. In some aspects, diabetes-induced decline in eGFR can be reversed by about 50%, by about 75%, or by about 100% following administration of compositions disclosed herein.

Compositions disclosed herein may reverse the diabetes-induced decline in urinary creatinine clearance compared to declining urinary creatinine clearance in an untreated diabetic subject with identical disease condition and predicted outcome. In some aspects, diabetes-induced decline in urinary creatinine clearance can be reversed by about 20% to about 40% following administration of compositions disclosed herein.

Compositions disclosed herein may reverse the diabetes-induced decline in renal blood flow compared to declining renal blood flow in an untreated diabetic subject with identical disease condition and predicted outcome. In In some aspects, diabetes-induced decline in renal blood flow can be reversed by at least about 25% following administration of compositions disclosed herein.

Compositions disclosed herein may prevent diabetic loss of podocytes compared to diabetic loss of podocytes in an untreated diabetic subject with identical disease condition and predicted outcome. In some aspects, the rate of diabetes-induced loss of podocytes can be decreased by at least about 50%, following administration of compositions disclosed herein.

Compositions disclosed herein may increase the expression of anti-oxidant markers in the diabetic kidney compared to the expression of anti-oxidant markers in an untreated subject with identical disease condition and predicted outcome. Non-limiting examples of anti-oxidant markers include SOD1 (superoxide dismutase 1), SOD2 (superoxide dismutase 2), CAT (catalase), GPX1 (glutathione peroxidase 1), and GPX3 (Glutathione peroxidase 3). In some embodiments, the expression of anti-oxidant markers following administration of compositions disclosed herein may be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other embodiments, the expression of anti-oxidant markers following administration of compositions disclosed herein may be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In additional embodiments the expression of anti-oxidant markers following administration of compositions disclosed herein may be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

Compositions disclosed herein may increase the expression of angiogenesis markers in the diabetic kidney compared to the presence of angiogenesis markers in an untreated subject with identical disease condition and predicted outcome. Non-limiting examples of angiogenesis markers include VEGF-A (vascular endothelial growth factor-A), PECAM1 (Platelet And Endothelial Cell Adhesion Molecule 1), eNOS (endothelial NOS), and KDR (vascular endothelial growth factor receptor 2). In some embodiments, the expression of angiogenesis markers following administration of compositions disclosed herein may be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other embodiments, the expression of angiogenesis markers following administration of compositions disclosed herein may be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In additional embodiments, the expression of angiogenesis markers following administration of compositions disclosed herein may be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In various embodiments, compositions disclosed herein may improve diabetic life expectancy compared to the diabetic life expectancy of an untreated subject with identical disease condition and predicted outcome. As used herein, “diabetic life expectancy” is defined as the time at which 50 percent of subjects are alive and 50 percent have passed away. In some aspects, diabetic life expectancy may be indefinite following treatment with a composition disclosed herein. In other aspects, diabetic life expectancy may be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some aspects, diabetic life expectancy may be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In additional aspects, diabetic life expectancy may be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

Compositions disclosed herein may revascularize a kidney. As used herein, the term “revascularization” refers to restoration of perfusion to a part of or a whole kidney that has suffered ischemia. In some aspects, compositions disclosed herein may revascularize a kidney that has suffered from at least 5%, at least 10%, at least 25%, at least 50%, at least 75%, or at least 99% ischemia. In other aspects, compositions disclosed herein may revascularize a kidney that has suffered from at least 5%, at least 10%, at least 25%, at least 50%, at least 75%, or at least 99% ischemia caused by diabetes. In various embodiments, a kidney can be revascularized by about 5%, by about 10%, by about 25%, by about 50%, by about 75%, or by about 100% following administration of compositions disclosed herein. In other embodiments, a diabetic kidney can be revascularized by about 5%, by about 10%, by about 25%, by about 50%, by about 75%, or by about 100% following administration of compositions disclosed herein. In some embodiments, compositions disclosed herein may revascularize a kidney by secretion of one or more paracrine factors. In some aspects, one or more paracrine factors comprises VEGF (vascular endothelial growth factor), VEGF-A (vascular endothelial growth factor A), VEGF-B (vascular endothelial growth factor B), bFGF (basic fibroblast growth factor), Ang-1 (Angiopoietin-1), Ang-2 (Angiopoietin-2), IL-1 (interleukin 1), IL-6 (interleukin 6), IL-1β (interleukin 1 beta), TNF-α (tumor necrosis factor alpha), TGF-β (transforming growth factor beta 1), SDF1 (stromal cell-derived factor 1), SDF-1α (stromal cell-derived factor 1 alpha), PIGF (placental growth factor), IGF-1 (insulin like growth factor 1), MCP-1 (monocyte chemoattractant protein-1), FGF-2 (fibroblast growth factor 7), FGF-7 (fibroblast growth factor 7), PDGF (platelet-derived growth factor), MMP-9 (matrix metalloproteinase 9), TB4 (Thymosin-beta-4), Sfrp (Secreted frizzled-related protein 1), Tenacin C, Thrombospondin-1, HGF, (Hepatocyte growth factor), or a combination thereof In other embodiments, compositions disclosed herein may revascularize a kidney by tube formation of the compositions disclosed herein. As used herein, the term “tube formation” refers to the formation of a network of capillary-like tube structures formed from and comprising EPCs. In some aspects, compositions disclosed herein revascularize a kidney by at least 5%, at least 10%, at least 20%, at least 50%, at least 75%, at least 99% by tube formation.

### (III) Methods of Using Compositions

Other embodiments of the present disclosure are methods of administering compositions disclosed herein to a subject in need wherein administration treats diabetic kidney disease. Still other embodiments of the present disclosure are methods of administering compositions disclosed herein to a subject in need wherein administration at least one symptom of diabetic kidney disease is improved by at least 25% within one month after administration.

(a) Methods of Administration

In various embodiments, compositions disclosed herein may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In other aspects, administration of the disclosed compositions by parenteral injection may be by slow or bolus methods as known in the field. In some embodiments, the route of administration by parenteral injection can be determined by the target location. In some aspects, compositions disclosed herein may be administered intrarenally. In other aspects, compositions disclosed herein may be administered under at least one kidney capsule of a subject in need thereof In still other aspects, compositions disclosed herein may be administered by retrograde injection through the ureter.

In various embodiments, the dose of compositions disclosed herein to be administered are not particularly limited, and may be appropriately chosen depending on conditions such as a purpose of preventive and/or therapeutic treatment, a type of a disease, the body weight or age of a subject, severity of a disease and the like. In other embodiments, administration of a dose of a composition disclosed herein may comprise a therapeutically effective amount of the composition disclosed herein. As used herein, the term “therapeutically effective” refers to an amount of administered composition that treats diabetic kidney disease, reduces proteinuria, reverses/prevents renal fibrosis, increase the diabetic-induced decline in eGFR, increase the diabetic-induced decline in urinary creatinine clearance, reverses the diabetic-induced decline in renal blood flow, prevents diabetic-induced podocyte loss, or a combination thereof.

A therapeutically effective amount of a composition disclosed herein to be delivered to a subject may be an amount that does not result in undesirable systemic side effects. In various embodiments, compositions administered as disclosed herein may comprise about 5% to about 95%, about 15% to about 85%, or about 25% to about 75% total genetically modified endothelial progenitor cells by total weight of the composition. In other embodiments, compositions administered as disclosed herein may comprise about 5% to about 95%, about 15% to about 85%, or about 25% to about 75% total genetically modified endothelial progenitor cells with transiently reduced p53 expression by total weight of the composition. In still other embodiments, compositions administered as disclosed herein may comprise about 5% to about 95%, about 15% to about 85%, or about 25% to about 75% total genetically modified endothelial progenitor cells with transiently reduced anti-apoptosis factor expression by total weight of the composition. In yet other embodiments, compositions administered as disclosed herein may comprise about 5% to about 95%, about 15% to about 85%, or about 25% to about 75% total genetically modified endothelial progenitor cells with transiently increased mitochondrial antioxidant expression by total weight of the composition. In other embodiments, compositions administered as disclosed herein may comprise about 5% to about 95%, about 15% to about 85%, or about 25% to about 75% total genetically modified endothelial progenitor cells with transiently increased MnSOD expression by total weight of the composition.

(b) Frequency of Administration

In some embodiments, a composition disclosed herein may be administered to a subject in need thereof once. In some embodiments, a composition disclosed herein may be administered to a subject in need thereof more than once. In other embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition may be administered to an subject in need thereof can depend on the discretion of a medical professional, the severity of the diabetic kidney disease, and the subject's response to the formulation. In some embodiments, a composition disclosed herein may be administered continuously; alternatively, the dose of drug being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In another aspect, dose reduction during a drug holiday may be from 10%-100%, including by way of example only 10%, 25%, 50%, 75%, and 100%.

In various embodiments, the desired daily dose of compositions disclosed herein may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once about 4 weeks.

In some embodiments, a composition as disclosed may be initially administered followed by a subsequent administration of one for more different compositions or treatment regimens. In other embodiments, a composition as disclosed may be administered after administration of one for more different compositions or treatment regimens.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

Generation of mouse diabetic animal models and transiently silenced EPCs for subcapsular bolus injection under the kidney capsule. An animal model of type 1 diabetes mellitus (DM1) was induced in healthy, non-obese C57B¹/₆J mice by serial streptozotocin (STZ) injections. First, tail vein blood glucose level was measured (Contour Next Blood Glucometer, Bayer) at day 0 after 6 hours of fasting before in 15 male mice and 15 female mice. Next, STZ (40 mg/kg bodyweight) was administered to each mouse by intra-peritoneal (IP) injection daily for 3-5 consecutive days. After 6-8 weeks post-STZ-injection, mouse tail vein blood glucose was measured to verify stable hyperglycemia (above 250 mg/dL). Mice with stable hyperglycemia are deemed diabetic and are further used as an animal model of DM1. Alternative models of diabetes suitable for experimentation include diet-induced obesity (DIO) mice (a model of pre-diabetes/type 2 diabetes mellitus (DM2)) and db/db mice (a model of DM2).

Mature endothelial cells (ECs), unlike endothelial progenitor cells (EPCs), are more resistant to p53-mediated apoptotic damage in presence of hyperglycemia. To prepare EPCs for use as a diabetic therapy, EPCs must therefore be protected from apoptosis in hyperglycemia in order to for the cells to mature into ECs, which usually takes 4 weeks. Herein, EPCs are protected from apoptosis by transient, rather than permanent, knock-down of p53 ex vivo. This is specifically accomplished by the application of an adenovirus as a gene silencer which has a transient expression of approximately 4 weeks. Specifically, mouse endothelial progenitor cells (mEPCs, defined as CD34-positive cells) were obtained fresh from peripheral blood mononuclear cells (PBMCs). Briefly, mouse PBMCs were isolated from circulating whole blood (collected from 3 mice in each category) by the Ficoll separation method. These cells were then cultured in Endothelial Cell Growth Medium-2 (EGM-2) for 5 to 7 days and trypsinized to collect mEPCs. After isolation of mEPCs, cells were cultured in StemSpan Serum-Free Expansion Medium II (SFEM II) for 5 days. Cells were subsequently transduced by ex vivo with Ad-Null or Adp53sh virus at 100 multiplicity of infection and then kept in culture for another 4 to 5 days. Adp53sh is an adenovirus used as a gene silencer for p53 which has a transient expression of approximately 4 weeks. Ad-Null is an adenovirus with no gene silencer that was used for control treatment of mEPCs.

After ex vivo transduction with wither Adp53sh or Ad-Null, transduced EPCs were delivered bilaterally under both kidney capsules, in each STZ-injected mouse by subcapsular bolus injection. Specifically, a small portion of the capsule of the mouse kidney was gently lifted from the kidney, using thumb forceps. Using a tuberculin syringe with a 33 gauge needle, one million p53-silenced or null EPCs (resuspended in 100 μl cell culture media), and control solution (100 μl saline) were injected into the renal subcapsular space. A drop of surgical glue was placed over the puncture site as the needle was retracted to seal-in the infused solution and prevent backflow. This surgical procedure generally required 20-30 minutes per mouse. During the surgical procedure, mouse vital signs were monitored every 5 minutes.

### Example 2

Transplantation of p53-silenced endothelial progenitor cells prevents progressive proteinuria in a diabetic animal model. Proteinuria is universal marker for progressive renal disease, and is viewed as a measure of the severity and determinant for diabetic renal disease progression. To determine if p53-silenced EPCs can reverse diabetic-associated proteinuria, STZ-induced diabetic mice and healthy mice were individually housed in metabolic cages to collect urine output over 24 hours. After baseline urine output was collected, diabetic mice were transplanted with either Ad-Null transduced EPCs or Adp53sh transduced EPCs bilaterally under the kidney capsule. Mice were returned to metabolic cages and 24-hour urine collection was performed at weeks 1, 2, 3, and 4 after EPC transplantation. Protein was measured from each 24-hour urine collection with a Randox RX Monza chemical analyzer.

Among the three groups of mice observed, proteinuria was reduced in diabetic mice one week after transplant of Adp53sh-m EPCs compared to AdNull-m EPC transplanted diabetic mice (FIG. 1). After 2 weeks, Adp53sh-m EPCs transplanted diabetic mice had no measured proteinuria, matching the protein urine amounts of healthy mice (FIG. 1). AdNull-m EPC transplanted diabetic mice; however, had increasing amounts of urinary protein over time, marking the untreated progression of diabetic kidney damage.

### Example 3

Transplantation of p53-silenced endothelial progenitor cells reduces excessive urine and plasma creatinine levels in a diabetic animal model. Excessive urinary and plasma creatinine levels are a hallmark of declining renal function in the diabetic kidney. To determine if p53-silenced EPCs can preserve and/or improve renal function in DM1, STZ-induced diabetic mice and healthy mice were individually housed in metabolic cages to collect urine output over 24 hours. After baseline urine output was collected, diabetic mice were transplanted with either Ad-Null transduced EPCs or Adp53sh transduced EPCs bilaterally under the kidney capsule. Mice were returned to metabolic cages and 24-hour urine collection was performed at weeks 1, 2, 3, and 4 after EPC transplantation. Urinary creatinine was measured from each 24-hour urine collection with a Randox RX Monza chemical analyzer. Four weeks after EPC transplantation, plasma was collected from healthy mice and STX-injected mice transduced with either Ad-Null transduced EPCs or Adp53sh transduced EPCs. Plasma creatinine was measured from the collection at 4 weeks with a Randox RX Monza chemical analyzer.

In the STZ-diabetic model transplanted with Ad-Null transduced EPCs, urinary creatinine remained elevated at all collected time points compared to the urinary creatinine levels of healthy mice (FIG. 2A). Comparatively, urinary creatinine levels in the STZ-diabetic model before transplantation with Adp53sh transduced EPCs were reduced by almost half one week after transplantation with Adp53sh transduced EPCs (FIG. 2A). Further, after 2 weeks urinary creatinine levels in the STZ-diabetic model transplanted with Adp53sh transduced EPCs reached close the urinary creatinine levels of health, non-diabetic control mice (FIG. 2A). Similarly, plasma creatinine levels four weeks after EPCs transplantation were lowest in the STZ-diabetic model transplanted with Adp53sh transduced EPCs compared to diabetic mice transplanted with Ad-Null transduced EPCs (FIG. 2B). Collectively, data indicate that locally treating advanced diabetes with p53-silenced EPCs improves declining renal function in the face of advancing diabetes.

### Example 4

Transplantation of p53-silenced endothelial progenitor cells improves renal blood perfusion in a diabetic animal model. Advancing diabetes can damage the renal vasculature, affecting the blood flow into and out of the kidneys. Thus, identifying a mechanism to improve renal perfusion would be beneficial for diabetic kidney function. Here, STZ-induced diabetic mice were transplanted with either Ad-Null transduced EPCs or Adp53sh transduced EPCs bilaterally under the kidney capsule. Twenty-eight days after EPC transplantation, renal blood flow was measured in the transplanted diabetic mice with a Laser Doppler Perfusion Imager (LDPI) system.

Blood flow was increased in the Adp53sh EPC transplanted kidney compared to Ad-Null EPC transplanted kidney, at day 28, post cell transplantation (FIG. 3). Data indicate that locally treating advanced diabetes with p53-silenced EPCs improves improved renal perfusion in the diabetic kidney.

### Example 5

Transplantation of p53-silenced endothelial progenitor cells, but not mesenchymal stromal cells, improves diabetic kidney disease outcomes. Some literature exists on the use of mesenchymal stromal cells (MSCs), a type of progenitor cells, in the treatment of diabetic cardiovascular disease with limited long-term data [1, 2]. However, the positive effect of MSC cell therapy in cardio vascular disease appears to depend on the paracrine properties of MSCs [1-3]. Although, transplanting MSCs may be a feasible approach to treating diabetic kidney disease outcomes, doing so may also promote adverse renal-damage due to the MSC-induced paracrine stimulation of mesenchymal transformation of endothelial-like structures in the kidney.

To determine if renal transplantation of MSCs or p53-silenced EPCs is the preferred mechanisms of treating diabetes-induced kidney disease, kidneys of STZ-diabetic mice were transplanted with either Ad-Null transduced EPCs, Adp53sh transduced EPCs, or unmodified MSCs (0.5 million cells bilaterally transplanted under the kidney capsule). Mice were individually house in metabolic cages for 24-hour urine collection at weeks 1, 2, 3, and 4 after EPC or MSC transplantation. At 28-days after EPC or MSC transplantation, renal blood flow was measured with a Laser Doppler Perfusion Imager (LDPI) system. Next, plasma was collected from the EPC or MSC transplanted diabetic mice at 4 weeks after EPC or MSC transplantation. Creatine was measured in each plasma sample, and protein and creatine were measured in each 24-hour urine collection with a Randox RX Monza chemical analyzer.

Diabetic mice transplanted with MSCs have the same amount of urinary protein over the four week period after transplantation as diabetic mice transplanted with Ad-Null transduced EPCs (FIG. 4A). However, diabetic mice transplanted with Adp53sh transduced EPCs excreted almost 50% less urinary protein over the four week period after transplantation compared to the other two treatment groups (FIG. 4A). Thus, transplantation of p53-silenced EPCs, but not MSCs, improves diabetic-induced proteinuria.

Similarly, the protein-to-creatinine ratio of diabetic mice transplanted with Adp53sh transduced EPCs was significantly lower than the protein-to-creatinine ratios of diabetic mice transplanted with either Ad-Null transduced EPCs or MSCs (FIG. 4B). These data demonstrate that renal function is improved in diabetic mice following transplantation of 53-silenced EPCs; however, the decline in renal function as a consequence of progressing diabetes is unchecked in diabetic mice transplanted with MSCs.

Renal blood flow was increased in diabetic mice 28-days post transplantation Adp53sh transduced EPCs compared to diabetic mice transplanted with either Ad-Null transduced EPCs or MSCs (FIG. 4C). In fact, diabetic mice transplanted with MSCs have worse renal blood flow than the control-treated diabetic mice transplanted with Ad-Null transduced EPCs (FIG. 4C).

Collectively, the data demonstrate that poor renal outcomes associated with progressive diabetes (proteinuria, declining renal function, poor renal blood flow) can be corrected with local administration of p53-silenced EPCs. Further, data show that local administration of MSCs is not a viable option for treatment of diabetic kidney disease and, in fact, may exacerbate diabetic-induced damage to renal vasculature.

### Example 6

Transplantation of p53-silenced endothelial progenitor cells improves the pathological damage that occurs with diabetic kidney disease. The most consistent pathological features of diabetic kidney disease (either type 1 or type 2 diabetes) are capillary basement membrane thickening and diffuse and nodular glomerulosclerosis. Further, diabetic kidney disease is also associated with primary and secondary pathological changes in the vascular and tubulo-interstitial compartments, the severity of which exerts a strong influence on the rate of loss of renal function.

Kidneys of STZ-diabetic mice were transplanted with either Ad-Null transduced EPCs, Adp53sh transduced EPCs, or unmodified MSCs (0.5 million cells bilaterally transplanted under the kidney capsule). Kidneys were harvested from mice 28 days after EPCs were transplanted and paraffin embedded for histological processing. Parrafin sections of the kidneys of mice from each treatment group where stained with isolectin-β4, a vascular marker, and counterstained with hematoxylin to observe pathology of the harvested kidneys.

Isolectin-β4 staining of kidneys from diabetic mice transplanted with Adp53sh transduced EPCs showed increased vascularity (FIG. 5C) compared to isolectin-β4 staining of diabetic mouse kidneys transplanted with Ad-Null transduced EPCs (FIG. 5B), diabetic mouse kidneys transplanted with unmodified MSCs (FIG. 5D) or diabetic mouse kidneys injected with saline under the kidney capsule (FIG. 5A). Extreme nephrosclerosis (sclerotic kidney sections), hallmarked by shrunken glomeruli, were observed in diabetic mouse kidneys transplanted with unmodified MSCs (FIG. 5D) and diabetic mouse kidneys injected with saline under the kidney capsule (FIG. 5A). An intermediate improvement of nephrosclerosis was observed in diabetic mouse kidneys transplanted with Ad-Null transduced EPCs (FIG. 5B); however, kidneys from diabetic mice transplanted with Adp53sh transduced EPCs showed marked improvement of nephrosclerosis compared to the other treatment groups (FIG. 5C).

The data collectively show that locally treating diabetic kidney disease with p53-silenced EPCs improves and/or reverses the diabetes-induced pathological damage to the kidney. Surprisingly, transplantation of MSCs is not a viable treatment for pathological damage resulting from diabetic kidney disease, and may in fact worsen the damage.

### Example 7

Transplantation of p53-silenced endothelial progenitor cells increases mRNA gene expression levels of vascular markers in kidneys of diabetic animals. In diabetic nephrosclerosis, improved vascularization or re-perfusion of a diabetic sclerotic kidney is the primary goal. To quantify regeneration of the vasculature in the diabetic kidney, mRNA expression levels of the vascular markers (VEGF-A, PECAM1, eNOS and KDR) can be assessed where increased expression of these markers suggests vasculature regeneration.

Kidneys of STZ-diabetic mice were transplanted with either Ad-Null transduced EPCs, Adp53sh transduced EPCs, or unmodified MSCs (0.5 million cells bilaterally transplanted under the kidney capsule). Kidneys were harvested from mice 28 days after EPCs were transplanted and processed for RNA extraction. Extracted RNA was subjected to qRT-PCR using standard methodology.

Endothelial NOS (eNOS) expression was increased almost 4-fold in kidneys harvested from diabetic mice transplanted with Adp53sh transduced EPCs compared to eNOS expression in the kidneys of diabetic mice transplanted with Ad-Null transduced EPCs (FIG. 6A). Vascular endothelial growth factor-A (VEGF-A) gene expression was also elevated in kidneys harvested from diabetic mice transplanted with Adp53sh transduced EPCs compared to VEGF-A expression in the kidneys of diabetic mice transplanted with Ad-Null transduced EPCs (FIG. 6A); however, vascular endothelial growth factor receptor 2 (KDR) levels were unchanged between the two treatment groups (FIG. 6A).

Kidneys harvested from diabetic mice transplanted with unmodified MSCs did not show a change in eNOS, VEGF-A, or KDR gene expression compared to the kidneys of control, diabetic mice transplanted with Ad-Null transduced EPCs (FIG. 6B). Accordingly, local administration of MSCs does not promote regeneration of the vasculature in the diabetic kidney.

### Example 8

Hyperglycemic conditions promote oxidative stress and suppress antioxidant gene expression. Formation of reactive oxygen species (ROS) or changes in ROS production can occur in the diabetic kidney with a broad range in effects. For example, oxidative stress can alter renal blood flow, increase renal tissue inflammation, promote fibrotic changes within the kidney, and increase the likelihood of proteinuria. To determine the effect of hypercalcemia alone, in vitro studies measured the mRNA gene expression levels of anti-oxidant markers (SOD1, SOD2, CAT, GPX1, and GPX3) in mature human, endothelial cells (HUVECS) either (1) exposed to high glucose (20 mM) for 28 days (HG) or (2) not exposed to glucose (NG). The cells were then harvested, RNA extracted, and subjected to qRT-PCR. Under hyperglycemic conditions, mRNA gene expression of SOD1, SOD2, CAT, GPX1, and GPX3 was reduced compared to gene expression levels in HUVECS not exposed to high glucose (FIG. 7). Cells under hyperglycemic conditions were also assessed for mRNA gene expression levels of the angiogenesis markers, VEGF-A, KDR, eNOS, and Platelet And Endothelial Cell Adhesion Molecule 1 (PECAM1). Compared to HUVECS not exposed to glucose, mRNA expression of VEGF-A, KDR, eNOS, and PECAM1 was decreased in HUVECS grown in high glucose for 28 days (FIG. 7).

Results in this study indicate that, in addition to improving survival of EPCs in a diabetic/hyperglycemic milieu by silencing p53, the function of EPCs can be improved by upregulating antioxidant MnSOD or SOD2 expression in the cells by co-transduction of EPC with Adp53sh and AdSOD2, sequentially. Therefore, it is expected that SOD2 upregulation in p53 silenced EPC has additive functional improvement when tested in the STZ-induced DM1 mouse model and in the DM2 db/db mouse model.

### REFERENCES CITED IN EXAMPLES

1. Griffin T P, Martin W P, Islam N, O'Brien T, Griffin M D. The promise of mesenchymal stem cell therapy for diabetic kidney disease. Curr Diab Rep. 2016; 16(5):42.

2. Ni W, Fang Y, Xie L, Liu X, Shan W, Zeng R, Liu J, Liu X. Adipose-derived mesenchymal stem cells transplantation alleviates renal injury in streptozotocin-induced diabetic nephropathy. J Histochem Cytochem. 2015; 63(11):842-53.

3. Ezquer M E, Ezquer F E, Arango-Rodriguez M L, Conget P A. MSC transplantation: a promising therapeutic strategy to manage the onset and progression of diabetic nephropathy. Biol Res. 2012; 45(3):289-96.

What is claimed is:

1. A method of treating diabetic kidney disease, the method comprising transplanting an endothelial progenitor cell genetically modified to have transiently reduced p53 expression under at least one kidney capsule of a subject in need thereof.

2. The method of claim 1, wherein the transiently reduced p53 expression is reduced for about 2 weeks to about 12 weeks.

3. The method of claim 1, wherein the transiently reduced p53 expression is reduced for at least about 4 weeks.

4. The method of claim 1, wherein the endothelial progenitor cell is derived from human CD34⁺ mononuclear cells.

5. The method of claim 1, wherein the endothelial progenitor cell expresses p53-specific siRNA or p53-specific shRNA.

6. The method of claim 1, wherein the endothelial progenitor cell genetically modified to have transiently reduced p53 expression is transplanted under at least one kidney capsule of a subject in need thereof at least about once a month.

7. The method of claim 1, wherein the subject in need thereof has type 1 diabetes mellitus, type 2 diabetes mellitus, or is pre-diabetic.

8. The method of claim 1, wherein the subject in need thereof has at least one symptom of diabetic kidney disease.

9. The method of claim 8, wherein the at least one symptom of diabetic kidney disease is proteinuria, renal fibrosis, at least a 25% decrease in estimated glomerular filtration rate (eGFR) compared to eGFR of a non-diabetic subject, at least a 25% decrease in urinary creatinine clearance compared to urinary creatinine clearance of a non-diabetic subject, at least a 25% decrease in renal blood flow compared to renal blood flow of a non-diabetic subject, at least a 25% loss of podocytes compared to the amount of podocytes of a non-diabetic subject, or a combination thereof.

10. The method of claim 8, wherein the at least one symptom of diabetic kidney disease is improved by at least 1% within one month after transplanting an endothelial progenitor cell genetically modified to have transiently reduced p53 expression under at least one kidney capsule.

11. The method of claim 1, wherein the endothelial progenitor cell genetically modified to have transiently reduced p53 expression is also genetically modified to have transiently increased expression of at least one mitochondrial antioxidant.

12. The composition of claim 11, wherein the at least one mitochondrial antioxidant comprises manganese superoxide dismutase (MnSOD).

13. The method of claim 1, wherein the method comprising transplanting an endothelial progenitor cell genetically modified to have transiently reduced p53 expression under at least one kidney capsule of a subject in need thereof increases the expression of at least one anti-oxidant marker or at least one angiogenesis marker.

14. A formulation suitable for injection under a kidney capsule, the formulation comprising at least one endothelial progenitor cell genetically modified to have transiently reduced p53 expression and at least one physiologically acceptable carrier.

15. The formulation of claim 14, wherein the transiently reduced p53 expression is reduced for about 2 weeks to about 12 weeks.

16. The formulation of claim 14, wherein the endothelial progenitor cell is derived from human CD34⁺ mononuclear cells.

17. The formulation of claim 14, wherein the endothelial progenitor cell expresses p53-specific siRNA or p53-specific shRNA.

18. The formulation of claim 14, wherein the endothelial progenitor cell is obtained from peripheral blood, umbilical cord blood, or bone marrow of a subject.

19. The formulation of claim 18, wherein the endothelial progenitor cell obtained is autologous to the subject.

20. The formulation of claim 18, wherein the formulation further comprises at least one pharmaceutically acceptable excipient.


SEQUENCE LISTING (2 sequences)

SEQ ID NO: 1 - DNA, 64 bp, Artificial Sequence
  synthetic sequence
gatccccgactccagtggtaatctacttcaagagagtagattaccactggagtctttttggaaa

SEQ ID NO: 2 - DNA, 64 bp, Artificial Sequence
  synthetic sequence
agcttttccaaaaagactccagtggtaatctactctcttgaagtagattaccactggagtcggg